(19) United States
(12) Patent Application Publication
(10) Pub. No.: US 2023/0277461 A1
(43) Pub. Date: Sep. 7, 2023

(54) OXYGEN-CONTAINING PARTICLES, MANUFACTURING METHOD THEREFOR, AND USE THEREOF

(71) Applicant: SUZHOU YIBEN LIFE TECHNOLOGY CO., LTD, Suzhou (CN)

(72) Inventors: Yonghua DONG, Solon, OH (US); Yuan HE, Suzhou (CN)

(73) Assignee: SUZHOU YIBEN LIFE TECHNOLOGY CO., LTD, Suzhou, JS (CN)

(21) Appl. No.: 18/174,549

(22) Filed: Feb. 24, 2023

Related U.S. Application Data
(63) Continuation of application No. PCT/CN2021/114376, filed on Aug. 24, 2021.

(30) Foreign Application Priority Data
Aug. 24, 2020 (CN) 202010855901.4

(51) Int. Cl. A61K 9/16; A61K 33/00; A61K 9/00

(52) U.S. Cl. CPC A61K 9/1682; A61K 33/00; A61K 9/0026

(57) ABSTRACT

Disclosed are oxygen-containing particles, a manufacturing method therefor, and a use thereof. The oxygen-containing particles comprise a carrier that is safe for the human body and has a large number of micropores or pores distributed on the surface. The oxygen-containing particles further comprise a peroxide crystal attached to the surface of the micropores or pores of the carrier. The oxygen-containing particles in the present invention have the advantage of having microparticles in addition to a slow and controllable release of oxygen. The oxygen-containing particles can achieve local or systemic oxygen supply during embolization, implantation, perfusion and other operations. Furthermore, the oxygen-containing particles can also be used alone to supply oxygen for patients with pulmonary dysfunction.

### BACKGROUND

### Technical Field

The disclosure relates to oxygen-containing particles, a manufacturing method therefor, and use thereof, belonging to the technical field of medical materials.

### Related Art

The oxygen-containing particles have wide application in medicine. For example, existing experimental data prove that: Urea peroxide used in cardiovascular contrast echocardiography has a good developing effect. The postoperative diagnosis is consistent with the results of contrast echocardiography, and the urea peroxide is safe. In addition, some people have proposed to use oxygen-containing particles to release oxygen at tumor sites to inhibit tumor, and others have proposed to use oxygen-containing particles to prepare photodynamic therapy agents. However, most of these oxygen-containing particles are hemoglobin and/or perfluorocarbons, and has great limitations in the scope of application.

### SUMMARY

The primary technical problem to be solved by the disclosure is to provide oxygen-containing particles.

Another technical problem to be solved by the disclosure is to provide a manufacturing method for the oxygen-containing particles.

Another technical problem to be solved by the disclosure is to provide a use of the oxygen-containing particles.

In order to realize the above objectives, the disclosure adopts the following technical solutions:

According to the first aspect of embodiments of the disclosure, provided are oxygen-containing particles including carriers that are safe to the human body and have numerous micropores or pores distributed in the surfaces, and further including peroxide crystals attached to the surfaces of the micropores or pores of the carriers.

According to the second aspect of embodiments of the disclosure, provided is a manufacturing method for the oxygen-containing particles, including the following steps:
S1: drying and sending the peroxide crystals to a drying room of fluidized bed equipment;
S2: delivering the prepared carriers that are safe to the human body to a charging room of the fluidized bed equipment, wherein numerous micropores or pores are distributed in the surfaces of the carriers,
S3: mixing the peroxide crystals with the carriers in a settling room of the fluidized bed equipment to make the peroxide crystals adhere to the surfaces of the micropores or pores of the carriers; and
S4: performing separation through a cyclone separator to obtain the oxygen-containing particles.

According to the third aspect of embodiments of the disclosure, provided is a manufacturing method for the oxygen-containing particles, including the following steps:
preparing a peroxide solution;
adding microspheres and completely immersing the microspheres in the peroxide solution;
cooling to room temperature to make the peroxide crystallize on the microspheres; and
separating the oxygen-containing microspheres, washing and drying the oxygen-containing microspheres to obtain the oxygen-containing particles.

According to the fourth aspect of embodiments of the disclosure, provided are oxygen-containing particles used for injection into blood vessels to achieve oxygen-containing embolization.

Or, provided are oxygen-containing particles used to detect arteriovenous fistula before intraarterial interventional radionuclide therapy.

Or, provided are oxygen-containing particles used to evaluate the optimal dosage of radioactive microspheres.

Or, provided are oxygen-containing particles used for implantation into tissues to achieve oxygen-containing implantation, where the oxygen-containing particles react with body fluid in the tissues to release oxygen.

Or, provided are oxygen-containing particles used for injection into blood vessels to achieve local oxygen supply.

The oxygen-containing particles provided by embodiments of the disclosure can be used for injection into blood vessels to achieve oxygen-containing embolization; used to for implantation into tissues to achieve oxygen-containing implantation; used to detect arteriovenous fistula before intraarterial interventional radionuclide therapy; used to evaluate the optimal dosage of radioactive microspheres, where the oxygen-containing particles react with body fluid in the tissues to release oxygen; and used for injection into blood vessels to achieve local oxygen supply.

Moreover, the oxygen-containing particles provided by embodiments of the disclosure have both the advantages of particles (such as microspheres, microcapsules, seed strands) and the advantages of slow and controllable release of oxygen, and can realize local or systemic oxygen supply while performing operations such as embolization, implantation, and perfusion. The oxygen-containing particles can also be used alone for oxygen supply to patients with pulmonary dysfunction.

### BRIEF DESCRIPTION OF THE DRAWINGS

FIG. 1 is a schematic structural diagram of oxygen-containing particles according to embodiments of the disclosure.

FIG. 2 is another schematic structural diagram of the oxygen-containing particles according to embodiments of the disclosure.

FIG. 3 is a flowchart of a manufacturing method for the oxygen-containing particles according to embodiments of the disclosure.

### DETAILED DESCRIPTION

The technical solutions of the disclosure are described below in detail in conjunction with the drawings and specific embodiments.

Conventional peroxide crystals include but are not limited to urea hydrogen peroxide [CO(NH2)2·1.5H2O2] crystals, sodium carbonate hydrogen peroxide (Na2CO3·1.5H2O2) crystals or sodium peroxide crystals. The urea hydrogen peroxide crystals are non-toxic, odorless white crystalline powder with dual properties of urea and hydrogen peroxide. The sodium carbonate hydrogen peroxide crystals are white rod-like crystals or crystalline powder. This is also analogous to sodium peroxide crystals. The shape and size of these crystals cannot be precisely controlled.

The disclosure first provides particle-like oxygen-containing particles with controllable shape and size. The so-called particles are small particles having numerous micropores or pores distributed in the surfaces of carriers that are safe to the human body, and drugs are adsorbed or dispersed on the surfaces of the micropores or pores. Microspheres with particle size of micron (e.g., 1-300 μm), seed strands with size of millimeter (e.g., 0.8 mm*4.5 mm), and microcapsules with size of micron (e.g., 50-500 μm), and the like are included. There are many carrier materials for preparing particles, mainly including natural polymer microspheres (such as starch microspheres, albumin microspheres, resin microspheres, gelatin microspheres, and chitosan) and synthetic polymer microspheres (such as polylactic acid microspheres). Among them, micron-sized particles such as microspheres or microcapsules are used for injection into blood vessels; and millimeter-sized particles such as seed strands are used for implantation into human tissues.

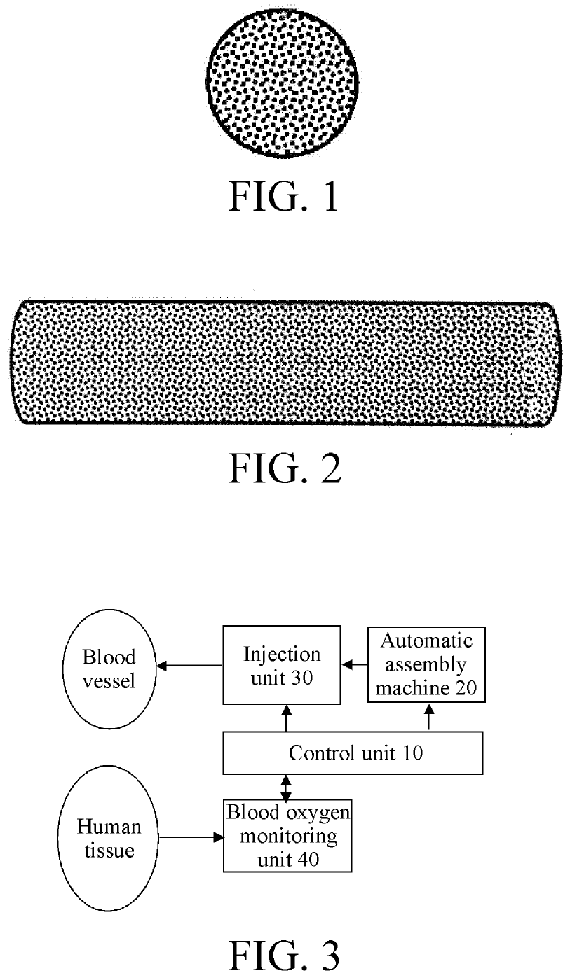

The oxygen-containing particles provided by embodiments of the disclosure are in the form of microspheres (FIG. 1) or seed strands (FIG. 2) using carriers with controllable shape and size, and have peroxide crystals such as urea peroxide uniformly adsorbed or dispersed thereon. Those skilled in the art can understand that, not limited to peroxide crystals, it may also be other substances that can generate oxygen in the human body. The carriers of the oxygen-containing particles have many shapes, such as microspheres or seed strands, but their size range is small, that is, they have a uniform size. The size of the carriers can vary with the needs of application scenarios, with controllable size range. For example, the carriers that are microspheres with a diameter of 20 μm are injected into the renal artery, or the carriers that are microspheres with a diameter of 50 μm are injected into the hepatic artery, or the carriers that are 0.8 mm*4.5 mm seed strands are implanted in the breast tissues.

The carrier may be prepared by may methods such as chemical synthesis, fluidized bed spraying or solvent extraction. The size and distribution density of micropores or pores in the surface of the carrier may be achieved by setting parameters in the preparation process. This is a conventional technology in the art.

According to the oxygen-containing particles provided by embodiments of the disclosure, the peroxide crystals are attached into the particles. In other words, as soon as the oxygen-containing particles come into contact with liquid, the peroxide will start to react, and release oxygen, and the time at which the gas appears (the time at which the bubbles start to generate in the blood vessel intervention, the duration of the continuous generation of the bubbles, and the quantity (or concentration) of the bubbles can be controlled, and can be designed according to clinical needs), which is conducive to development and tracing. In addition, in order to continuously carry out examination operations such as B-ultrasound observation, or treatment operations such as oxygen-containing embolization, or systemic oxygen supply, it is necessary to continuously feed the oxygen-containing particles to continuously generate oxygen. B-ultrasound is very sensitive to bubbles in the body, and can also contrast small bubbles. Even though the peroxide on the three-dimensional surfaces of micron-sized particles releases oxygen, the amount of the peroxide attached to the particles is not large because of their small size, so a single particle only releases a small amount of oxygen, which shows aerosol development under B-ultrasound.

The application examples of the oxygen-containing particles provided by embodiments of the disclosure are described below.

### Application Example I: The Oxygen-Containing Particles are Injected into Blood Vessels for Oxygen-Containing Embolization

The oxygen-containing embolization of the disclosure refers to use of particles that can produce oxygen to provide oxygen while embolizing blood vessels.

Conventional arterial radioembolization will make tumor cells anoxic, and the anoxic state in residual cancer tissues will lead to significantly increased expression of VRGF and MVD compared with that before embolization, which will further lead to changes in blood supply of the residual cancer tissues and tumor neovascularization, so it is necessary to strengthen anti-angiogenesis treatment of tumors. Moreover, tumor tissues will “sleep” in case of hypoxia and ischemia, and is not sensitive to chemotherapy and radiotherapy, forming tolerance, leading to poor treatment effect.

The oxygen-containing particles provided by embodiments of the disclosure will generate oxygen when injected into the body. The carriers of the oxygen-containing particles (such as microspheres) will embolize target blood vessels. The peroxide attached to the oxygen-containing particles will release oxygen to the tumor cells, making the tumor cells ischemic but not hypoxic. This will not form a self-protection mechanism, and the tumor cells will still be sensitive to chemotherapy and radiotherapy. For example, taking the urea peroxide particles formed by urea peroxide crystals attached to the microsphere carriers as an example, when it is used as an oxygen-containing embolization, the urea peroxide particles are injected into the blood vessels, and the urea peroxide on the microspheres slowly releases oxygen in the blood, changing the state of tissue hypoxia, which will reduce the inflammatory reaction and neovascularization of tumor tissues after embolization, and the tolerance of tumor cells after oxygen inhalation to radiotherapy and chemotherapy.

### Application Example II: The Oxygen-Containing Particles are Used to Detect Arteriovenous Fistula Before Radionuclide Therapy

In intraarterial interventional radionuclide therapy, the nuclide emitting βparticles is mainly used at present. The commonly used radionuclides include ³²P, ⁹⁰Y, ¹³¹I, etc. It is required that radionuclides and carriers have high mechanical stability and high chemical stability, and the size of microspheres is 46-76 μm; and the radioactivity is 370-555 MBq (10-15 mCi), and the relative volume mass of microspheres is less than 2.5.

Treatment Method:
(1) The radioactive microspheres need to be prepared into suspension before injection. Due to the large proportion of the microspheres, glycerol should be added to make them suspend to ensure that the microspheres will not settle during injection.
(2) The arterial catheter should be inserted into the artery closest to the tumor as far as possible. For a superficial tumor, methylene blue can be injected through the catheter first to observe whether methylene blue is concentrated in the tumor site. For a deep tumor, ^{99m}TcS-colloid or ^{99m}Tc-MAA can be injected at the same time to observe whether there is radioactive concentration at the tumor site.
(3) The total dosage of the radioactive microspheres depends on the size of the tumor. Generally, it should ensure that the absorbed dose of tumor tissues reaches 60-100 Gy, so the total activity of the radioactive microspheres is 1.85-3.7 GBq (50-100 mCi); If the tumor is large, the activity can even reach 7.4 GBq (200 mCi).

At present, when injecting yttrium 90 microspheres to treat tumors, such as liver cancer, it is necessary to determine whether patients have arteriovenous fistula in the tumor and large shunt flow one day in advance. For such patients, intraarterial interventional radionuclide therapy is not suitable. Because hepatic arteriovenous fistula is always a complication of primary liver cancer, there is a fistula between the artery and the vein, which makes the blood in the artery flow into the vein through the confluent fistula, thus causing the radioactive microspheres injected into the hepatic artery to enter the vein and further enter the pulmonary circulation. Therefore, the hepatic arteriography is needed the day before operation, and non-radioactive isotopes are used for testing (for example, ^{99m}Tc-MAA with a particle size range close to ⁹⁰Y microspheres is widely used for pre-treatment evaluation of arteriovenous shunt to pulmonary system and hepatic perfusion of microspheres) to confirm whether there will be microspheres flowing into the vein to ensure safety.

Using the oxygen-containing particles provided by embodiments of the disclosure, such as urea peroxide microspheres, before the operation of perfusion of yttrium 90 microspheres (for a same operation, it is not needed to be performed one day in advance), the urea peroxide microspheres with the same size as the radioactive particles (or microspheres or microcapsules with size smaller than the size of the radioactive particles) are first injected from the artery, and then the positions of these urea peroxide microspheres under B-ultrasound are observed. If air bubbles (oxygen released by urea peroxide microspheres) are observed in the hepatic veins and inferior vena cava, it indicates that there is hepatic arteriovenous fistula, so the operation of perfusion of radioactive microspheres cannot be performed; and if no bubbles are observed, the operation of perfusion of radioactive microspheres can be immediately performed. In this way, the patient's arteriovenous fistula test and the operation of perfusion of radioactive microspheres are continuous, thus saving the time of doctors and patients, and also convenient for operation. Moreover, the oxygen-containing particles provided by embodiments of the disclosure can be used to detect the shunt, which can be checked by B-ultrasound, with low cost and no radiation.

At the same time, the distribution of the oxygen-containing particles in the body can also be observed through B-ultrasound, which is conducive to treatment.

### Application Example III: The Oxygen-Containing Particles are Used to Evaluate the Optimal Dosage of the Radioactive Microspheres

As described in the above application example II, the total dosage of the radioactive microspheres depends on the size of the tumor, but the shunt should also be considered. That is, not all the radioactive microspheres will enter and remain in the tumor tissues. Therefore, it is necessary to test the quantity of microspheres remained in the tumor tissues (or the proportion of microspheres to perfusion volume) by using the oxygen-containing particles that have no toxic side effects on the human body in advance. Then, the optimal dosage of the radioactive microspheres is estimated by using the quantity or proportion of microspheres, in combination with the size of the tumor.

For specific estimation methods, it can refer to Narsinh et al. “Hepatopulmonary Shunting: A Prognostic Indicator of Survival in Patients with Metastatic Colorectal Adenocarcinoma Treated with ⁹⁰Y Radioembolization”, Radiology, Vol. 282, No. 1, January 2017. It is proposed to calculate the pulmonary shunt rate by injecting a developer technetium-labeled polymeric albumin, ^{99m}Tc-MAA (99mTc is technetium-99 M isomer) at the same location before injecting ⁹⁰Y microspheres.

### Application Example IV: The Oxygen-Containing Particles are Injected into Artery for Local Oxygen Supply

In such cases as coronary heart disease, diabetes and other vascular diseases, cerebrovascular diseases, local microcirculatory disorders, local tissue edema, terminal artery stenosis, and the like, local hypoxia will occur, which will lead to pathological changes. By injecting the oxygen-containing particles provided by embodiments of the disclosure continuously into the corresponding artery, the symptoms caused by hypoxia can be relieved quickly. Because oxygen generated by peroxide contained in the blood, only a small amount of blood is needed to flow through the local ischemic tissues to meet the oxygen supply of local tissue cells, so as to improve cell metabolism and promote tissue repair.

### Application Example V: The Oxygen-Containing Particles are Injected into Vein for Systemic Oxygen Supply

The oxygen-containing particles provided by embodiments of the disclosure are continuously injected into the vein to improve the blood oxygen content of the whole body through venous blood circulation, which is suitable for respiratory attenuation, such as chronic obstructive pulmonary disease (COPD) and other blood circulation disorders.

### Application Example VI: The Oxygen-Containing Particles are Implanted into Tissues for Oxygen-Containing Implantation

The oxygen-containing particles provided by embodiments of the disclosure are implanted into the human tissues, such as breast or prostate tumor tissues. The peroxides react with body fluid in the tissues, release oxygen, and change the tolerance or neovascularization caused by hypoxia in the tumor tissues.

### Application Example VII: The Oxygen-Containing Particles Embolize Tumor Blood Vessels Under Ultrasound Induction

First, the site where tumor blood vessel embolism needs to be formed is located, the oxygen-containing particles are injected into the target blood vessels, under B-ultrasound development, the oxygen-containing particles for releasing oxygen microbubbles enter the tumor blood vessels, and then low-power ultrasound is used to localize and irradiate the site to induce the cavitation effect of the oxygen microbubbles at the site to form the tumor blood vessel embolism. On the one hand, the oxygen microbubbles embolize the tumor blood vessels and cut off their nutritional supply. On the other hand, the ultrasound causes the oxygen microbubbles to cavitate and burst, releasing oxygen molecules, providing oxygen for the tumor blood vessels, and maintaining or improving the oxygen content of local tissues, so that the tumor cells will not have tolerance to chemotherapy and radiotherapy.

### Application Example VIII: The Oxygen-Containing Particles Treat Vasospasm Under Ultrasound Induction

The oxygen-containing particles are injected into the target blood vessels through the vein to release the oxygen microbubbles. Then, high intensity mechanical index ultrasound is used to intervene the spastic vascular site, which causes the oxygen microbubbles to oscillate periodically and burst, resulting in “cavitation effect”, so as to achieve the purpose of improving vasospasm.

The oxygen-containing particles provided by embodiments of the disclosure have both the characteristics of particles (including microspheres, microcapsules, seed strands and other micron-level particles, or seed strands for implantation) and the characteristics of oxygen supply in vivo, so the oxygen-containing particles provided by embodiments of the disclosure can be used in most application scenarios of particles or oxygen supply in vivo. The above eight application examples are only examples of the application scenarios of the oxygen-containing particles provided by embodiments of the disclosure, and do not constitute a limitation to the disclosure. Those skilled in the art can understand that the oxygen-containing particles can also be used in other scenarios, for example, in combination with immune agents and vaccines to improve the effect.

In order to achieve continuous oxygen supply, an oxygen supply device as shown in FIG. 3 is used to feed the oxygen-containing particles into the human body (or animal). The oxygen supply device includes a control unit 10, an automatic assembly machine 20, an injection unit 30, and a blood oxygen monitoring unit 40. The control unit 10 controls the assembly time of the automatic assembly machine 20, the specification of assembly particles, the assembly speed, and the like, and provides the oxygen-containing particles of an appropriate size at the appropriate speed and time. The control unit 10 also controls the injection speed, injection position, and the like of the injection unit 30. The control unit 10 also controls the blood oxygen monitoring unit 40 to achieve the monitoring on the blood oxygen saturation, blood oxygen content and other indicators in the human body.

The automatic assembly machine 20 mixes an appropriate quantity of oxygen-containing particles with normal saline according to a set proportion, and then feeds the mixture to the injection unit 30. Since the oxygen-containing particles release oxygen slowly, even if the oxygen-containing particles react with the normal saline, the amount of oxygen released in a very short time will not affect the injection. Alternatively, the automatic assembly machine 20 mixes an appropriate quantity of oxygen-containing particles with normal saline (or other solvents that do not react with the peroxides and are also safe to the human body) according to a set proportion, and then injects the mixture into the body through the injection unit 30. The oxygen-containing particles contact with the blood in the body, and then slowly releases oxygen. According to the needs of patient conditions, the control unit 10 controls injection positions, i.e., the vein or the artery. For example, for diseases requiring local oxygen supply mentioned in application example IV, the oxygen-containing particles are injected into the artery. For the blood circulation disorders mentioned in application example V, the oxygen-containing particles are injected into the vein.

The manufacturing method for the oxygen-containing particles provided by embodiments of the disclosure are described below. It should be noted that, the following steps are only examples, and the sequence of steps or each step can be adjusted according to tactual situations.

S1: the peroxide crystals are dried and sent to a drying room of fluidized bed equipment.

The temperature and humidity in the drying room need to be set according to the nature of peroxide crystal materials fed in to keep the peroxide crystals dry.

S2: the prepared carriers are delivered to a charging room of the fluidized bed equipment.

The carriers need to be kept dry and uniform in specification.

S3: the peroxide crystals are mixed with the carriers in a settling room of the fluidized bed equipment.

Described in “Correlation of attached biomass concentration and biofilm thickness in a fluidized bioreactor”, Journal of Chemical Engineering of Chinese Universities, No. 02, 2019, in a fluidized bioreactor with negligible wall effects and containing regular slight-heavy particles as bio-carriers, biomass attachment on the carriers can be stably controlled by the fluidized bioreactor. It can be seen that peroxide crystals can be accurately and uniformly attached to particles with uniform specifications through the fluidized bed.

The pressure, temperature and humidity in the settling room are determined by the physical and chemical properties of the peroxide and the carrier.

S4: separation is performed through a cyclone separator to obtain the oxygen-containing particles.

Due to the high drying efficiency of the fluidized bed, the particles are suspended and dispersed in the air stream for attachment, which is suitable for attachment of tens or hundreds of micrometer-sized particles The microsphere-like or microcapsule-like oxygen-containing particles of the disclosure have a particle size range of 10-300 μm, including 20 μm, 50 μm, 80 μm, 100 μm, 150 μm, and 200 μm. The seed strand-like oxygen-containing particles has a particle size range of 0.5-1 mm and a length range of 10-60 mm, preferably 10 mm, 20 mm, and 50 mm.

The oxygen-containing particles provided by embodiments of the disclosure may also be attached to the micropores in the surfaces of the particles by surface recrystallization. The organic solvent dissolves the peroxide into liquid, which is mixed with the carriers (such as microspheres), and then dried to crystallize the peroxide into the carriers again.

Taking the urea peroxide microspheres as an example to illustrate the steps of preparing the oxygen-containing particles by the surface recrystallization.

S10: a peroxide solution is prepared at a temperature higher than room temperature

As mentioned by Yuan Wei et al of Beijing University of Chemical Technology in “Synthesis and application of urea peroxide”, Chemical Science and Technology, Vol. 7, No. 2, 1999, there are many methods to prepare a urea peroxide solution, including dry process and wet process, and wet process is commonly used to prepare the urea peroxide. In this method, a saturated or supersaturated urea solution is reacted with a hydrogen peroxide solution with a certain concentration, and the urea peroxide is obtained through crystallization, filtration, and drying.

In the disclosure, the saturated or supersaturated urea solution is reacted with the hydrogen peroxide solution with a certain concentration to obtain the urea peroxide solution by a conventional process.

For example, 60 g of urea and 200 g of hydrogen peroxide with a mass concentration of 25% are mixed at room temperature and stirred slightly to form a urea suspension. Then, the urea suspension is heated to completely dissolve urea to obtain the urea peroxide solution.

Here, the ratio of urea to hydrogen peroxide is only an example. Other ratios may be used according to needs, as long as it is ensured that in a next step, after heating to a certain temperature, the urea can be completely dissolved in the hydrogen peroxide.

For the selection of temperature, on the one hand, it needs to consider the corresponding relationship between the solubility of urea in hydrogen peroxide and temperature; and on the other hand, it needs to consider the material properties of the microspheres, and 45-100° C. is selected here.

S11: microspheres are added and completely immersed in the peroxide solution,

Here, the microspheres are 25-30-micron porous microspheres. It is well known that the smaller the particle size and the larger the specific surface area of the microspheres, the faster the release rate of the peroxide adsorbed on the microspheres. Therefore, the particle size of the microspheres is determined according to the actual application scenarios (for example, capillary or artery).

The quantity of the microspheres needs to be selected according to the preset release rate and concentration of the peroxide particles (urea peroxide), such as 200-500 g. After the microspheres are added to the urea peroxide solution, the microspheres are squeezed with a mesh cover, and the like, so that the microspheres are completely immersed in the urea peroxide solution, for example, the microspheres are pressed to the bottom of a container of the urea peroxide solution.

S12: the mixture is cooled to room temperature to make peroxides crystallize on the microspheres

Through temperature control, the temperature is reduced to room temperature, so that the urea peroxide is separated from the urea peroxide solution and attached to the surface or internal micropores or pores of the microspheres. At this time, the microspheres with peroxides (such as the urea peroxide), namely the oxygen-containing particles, have been formed.

S13: the oxygen-containing particles are separated out

Solids are poured out from the mixture at room temperature obtained in S12, and then the solids are slowly poured into a container containing ice water. Then, the solids are filtered with a 20-micron filter element and washed with the ice water to remove peroxide crystals on the surfaces of the oxygen-containing particles. Then, the solids filtered out of the filter element are put into a large amount of ice water, and then rotated in a sedimentation separator to remove sediments at the lower layer, and obtain the oxygen-containing particles from the upper layer. The separating operation is repeated many times to obtain all wet oxygen-containing particles.

S14: rinsing and drying are performed to obtain the oxygen-containing microspheres

The wet oxygen-containing particles are rinsed with 100 ml of ethanol several times, and subjected to suction filtration and vacuum drying to obtain dry oxygen-containing particles. The dry oxygen-containing particles are stored at low temperature.

The oxygen-containing particles provided by embodiments of the disclosure are preferably urea peroxide. The urea peroxide has high water solubility and good stability, and can be decomposed into hydrogen peroxide, urea, carbon dioxide, ammonia, and the like, which are harmless to the human body and can be discharged. The urea peroxide can be decomposed at room temperature, and the decomposition rate is slow and controllable (safer than hydrogen peroxide). Moreover, the hydrogen peroxide produced by the decomposition of the urea peroxide in the blood of the body can supply oxygen in the blood vessels and absorb carbon dioxide, thus replacing the oxygen supply in the lungs (venous oxygen supply). The oxygen is provided in the human tissues.

In order to improve the oxygen release rate, it may also be achieved by optimizing the carrier structure of the oxygen-containing particles. For example, the specific surface area of the carrier is changed to change the mass of the peroxides on the oxygen-containing particles per unit mass, thus changing the oxygen release rate. The release rate and quantity of oxygen may be also changed by controlling the automatic assembly unit to change the proportion of oxygen-containing particles and normal saline. Moreover, because the oxygen release rate of the urea peroxide is slow, before injection into the body, it is necessary to control the injection unit to inject the urea peroxide into the body at an appropriate time and an appropriate speed after the automatic assembly unit mixes the oxygen-containing particles with the normal saline in proportion.

The disclosure is described in detail above. For a person of ordinary skilled in the art, any obvious changes made to the disclosure without departing from the essence of the disclosure will constitute an infringement of the patent right of the disclosure and shall bear corresponding legal liabilities.

What is claimed is:

1. Oxygen-containing particles, comprising carriers that are safe to the human body and have micropores or pores distributed in the surfaces, and further comprising a peroxide crystal attached to the surfaces of the micropores or pores of the carriers.

2. A manufacturing method for the oxygen-containing particles according to claim 1, comprising the following steps:
S1: drying and sending the peroxide crystals to a drying room of fluidized bed equipment;
S2: delivering the prepared carriers that are safe to the human body to a charging room of the fluidized bed equipment, wherein micropores or pores are distributed in the surfaces of the carriers,
S3: mixing the peroxide crystals with the carriers in a settling room of the fluidized bed equipment to make the peroxide crystals adhere to the surfaces of the micropores or pores of the carriers; and
S4: performing separation through a separator to obtain the oxygen-containing particles; or comprising the following steps:
preparing a peroxide solution;
adding microspheres and completely immersing the microspheres in the peroxide solution;
cooling to room temperature to make the peroxide crystallize on the microspheres; and
separating the oxygen-containing microspheres, washing and drying the oxygen-containing microspheres to obtain the oxygen-containing particles.

3. (canceled)

4. A use of the oxygen-containing particles according to claim 1, wherein the oxygen-containing particles are used for injection into blood vessels to achieve oxygen-containing embolization; to detect arteriovenous fistula before intraarterial interventional radionuclide therapy; to evaluate the optimal dosage of radioactive microspheres; for implantation tissues to achieve oxygen-containing implantation, wherein the oxygen-containing particles react with body fluid in the tissues to release oxygen; or for injection into blood vessels to achieve local oxygen supply.

5-8. (canceled)
